(19) United States
(12) Patent Application Publication
(10) Pub. No.: US 2018/0280485 A1
(43) Pub. Date: Oct. 4, 2018

(54) THIORETINAMIDE COMPOSITIONS FOR THE APOPTOSIS OF MALIGNANT CELLS WHILE PREVENTING THE APOPTOSIS OF NORMAL CELLS AND RELATED METHODS

(71) Applicant: Kilmer McCully, Winchester, MA (US)

(72) Inventor: Kilmer McCully, Winchester, MA (US)

(21) Appl. No.: 15/475,103

(22) Filed: Mar. 30, 2017

(51) Int. Cl. A61K 38/54; A61K 35/39; A61K 38/48; A61K 38/46; A61K 31/16; A61K 31/07

(52) U.S. Cl. CPC A61K 38/54; A61K 35/39; A61K 31/07; A61K 38/465; A61K 31/16; A61K 38/4826

(57) ABSTRACT

The invention pertains to novel compositions and related method for stimulating the immune system to promote the apoptosis of malignant cells while inhibiting the apoptosis of normal cells by employing compositions of thioretinamide with organic allyl sulfides or furanonaphthoquinones and in particular diallyl trisulfide, napabucasin and optionally with pancreatin and/or cobalamin. The novel compositions and method are designed to work with the immune system to treat a wide variety of malignant neoplasms which involve working with an immune system or a reduced functioning of the immune system.

This invention pertains to compositions of thioretinamide, allyl sulfides, naphthoquinones, and pancreatic enzymes and related method of metabolic control utilizing the novel compositions to treat cancer, and a wide variety of neoplasms involving impaired functioning immune systems. The method combines administration of thioretinamide, allyl sulfides, especially diallyl trisulfide, and napththoquinones, especially napabucasin, with pancreatic enzymes for enzymatic degradation of homocysteinylated proteins, nucleic acids, and glycosaminoglycans, together with vitamins, amino acids and nitrilosides to enhance metabolic conversion of homocysteine thiolactone and retinol to thioretinamide by cystathionine synthase, to promote apoptosis of malignant cells and inhibit apoptosis of normal cells, and to promote synthesis of thioretinaco from thioretinamide and cobalamin in regenerative cells, thereby enhancing immune function and preventing the development and progression of cancer, and related immune system impairment.

### BACKGROUND OF THE INVENTION

Accumulation of homocysteine in tissues, cells and body fluids occurs in degenerative diseases of aging, including cancer, arteriosclerosis, osteoporosis, dementia, and autoimmune diseases, secondary to depletion of thioretinaco ozonide from mitochondria and cellular membranes, as described by McCully K S Comprehensive Physiology 2016; 6: 471-505. As taught in McCully U.S. Pat. No. 9,216,209, a method for control of abnormal accumulation of homocysteine in degenerative diseases of aging consists of administration of thioretinamide, together with enzymatic degradation of homocysteinylated proteins, nucleic acids, and glycosaminoglycans by pancreatic enzymes, and with vitamins, amino acids, and nitrilosides to enhance metabolic conversion of homocysteine and retinol to thioretinamide by cystathionine synthase and to promote synthesis of thioretinaco in regenerative cells and measuring and adjusting homocysteine levels, thereby ameliorating the development and progression of degenerative diseases.

Thioretinamide is formed by reaction of retinoic acid with homocysteine thiolactone, catalyzed by cystathionine synthase as reported by McCully K S Annals of Clinical and Laboratory Science 2011; 41: 301-314. Retinol is transported to cells by plasma transthyretin, and retinol is oxidized to retinoic acid by superoxide produced by the heme oxygenase function of cystathionine synthase, as described by McCully K S Comprehensive Physiology 2016; 6: 471-505. Two molecules of thioretinamide combine with cobalamin to produce thioretinaco. Ozone oxidizes the sulfur atoms of thioretinaco to sulfonium centers, forming the active site of thioretinaco ozonide. Binding of adenosine triphosphate (ATP) to the active site occurs because of binding of the alpha and gamma phosphate anions of ATP to the tetrahedral orbitals of the two sulfonium atoms of thioretinaco during the process of oxidative phosphorylation. Nicotinamide adenine dinucleotide and phosphate are the precursors of ATP, the formation of which is catalyzed by electron transport from electron transport complexes for reduction of oxygen to water during oxidative phosphorylation, as reported by McCully K S Annals of Clinical and Laboratory Science 2015; 45: 222-225. Thioretinaco ozonide catalyzes formation of adenosyl methionine from methionine, and adenosyl methionine regulates homocysteine production from methionine by allosteric inhibition of methylenetetrahydrofolate reductase and allosteric activation of cystathionine synthase.

The hyperhomocysteinemia observed in diseases of aging, including arteriosclerosis, cancer, osteoporosis, autoimmune diseases and dementia, is attributable to deficiency of adenosyl methionine, secondary to decreased synthesis of adenosyl methionine by thioretinaco ozonide. The aerobic glycolysis observed in cancer cells is attributed to deficiency of thioretinaco ozonide from mitochondria and cellular membranes, leading to synthesis of ATP by glycolysis instead of synthesis of ATP by oxidative phosphorylation. The increased growth rate, aggregation of nucleoproteins, degradation of cellular membranes, and abnormal oxidative metabolism observed in malignant cells are attributable to accumulation of homocysteine thiolactone within cells because of deficiency of thioretinaco ozonide.

In early childhood plasma homocysteine levels are low, approximately 2 to 6 μmol/L, increasing to adult levels of 8 to 10 μmol/L following puberty. Childhood malignancies, including leukemia, neuroblastoma, and neoplasms of developmental origin, are attributable to an immature, reduced or impaired immunity, according to the immune surveillance theory of cancer. In older adults, the incidence of malignant tumors is highly correlated with age and with elevated plasma homocysteine levels of 14 to 20 μmol. The elevation of plasma homocysteine levels in aging is attributable to declining cellular concentrations of thiorcinaco ozonide and adenosyl methionine, causing dysregulation of methionine metabolism through decreased allosteric inhibition of methylenetetrahydrofolate reductase and decreased allosteric activation of cystathionine synthase, as reviewed by McCully K S Comprehensive Physiology 2016; 6: 471-505. Also infection by pathogenic microbes causes alteration of homocysteine metabolism and suppressed immunity in atherosclerosis, as reviewed by McCully K S Frontiers in Aging Neuroscience 2017, In press. These observations suggest that impaired immune function in aging results from deficiency of thioretinaco ozonide, leading to decreased immune surveillance of dysplastic cells in aging and increased risk of malignant neoplasms.

The growth of human cancer cells in culture is strongly inhibited by atmospheric ozone levels of 0.3 to 0.9 ppm, whereas normal human diploid fibroblasts of lung origin are resistant to the inhibitory effect of ozone, as reported by Sweet F et al Science 1980; 209: 931-933. The cancer cells were derived from human broncho-alveolar adenocarcinomas, breast adenocarcinomas, uterine carcinosarcomas, and endometrial carcinomas. The inhibitory effect of ozone on growth of malignant cells may be related to deficiency of thioretinaco in malignant cells, causing decreased formation of thioretinaco ozonide and increased intracellular concentrations of free ozone, leading to cellular apoptosis. Experimental exposure to ozone causes apoptosis of rat hippocampus cells, as reported by Rodriguez-Martinez E et al Frontiers in Aging Neuroscience 2016; 245: 1-9, and exposure to ozone causes apoptosis of murine lung alveolar cells, as reported by Kirichenko A et al Toxicology and Applied Pharmacology 1996; 141: 416-424 and by Kosmider B et al Free Radicals in Biology and Medicine. 2010; 48: 1513-1524.

Antibodies have the capacity to destroy antigens by utilizing singlet oxygen, ¹O₂*, in the production of hydrogen peroxide from oxidation of water, as reported by Wentworth A D et al Proceedings of the National Academy of Sciences USA 2000; 97: 10930-10935. The reaction is considered to yield hydrogen trioxide (H₂O₃) as a key intermediate, and ozone is proposed to be produced by antibodies, as a means of killing bacteria, as discussed by Lerner R A et al Proceedings of the National Academy of Sciences USA 2003; 100: 3013-3015. Ozone is known to destroy hydrogen peroxide in a peroxone process responsible for killing of micro-organisms in water purification. By analysis of cholesterol ozonolysis products, evidence is presented for the presence of ozone in human atherosclerotic arteries, as reported by Wentworth P et al Science 2003; 302: 1053-1056. These studies implicate thioretinaco in the pathophysiology of immune reactions by its reaction with ozone to form thioretinaco ozonide.

Dendritic cells of the skin were identified by Langerhans in the 19^{th} century, and recent study of dendritic cells in multiple organs and tissues have documented the ability of these cells to present antigens effectively to activate natural killer (NK) lymphocytes. The ability of NK cells, which are activated by dendritic cells to cause degeneration and apoptosis of cancer cells, is the basis of the vaccination strategy of the immunotherapy of cancer, as discussed by Palucka K et al Nature Reviews Cancer 2012; 12: 265-277. According to this concept, tumor antigens are presented by dendritic cells to activated NK cells, which mediate immune-based killing of tumor cells by production of ozone, oxygen radicals, hypochlorite, and peroxynitrite. Exposure of pulmonary dendritic cells to ozone promotes allergic sensitization by a toll-like receptor (TLR4)-dependent mechanism, as reported by Hollingsworth J W et al Journal of Allergy and Clinical Immunology, 2010; 125: 1167-1170.

Multiple pathogenic microbes have been implicated in the pathogenesis of atherosclerosis and dementia, as reported by Ravnskov U, McCully K S American Journal of the Medical Sciences 2012; 344:391-394, and McCully K S Journal of Alzheimer's Disease 2016; 54: 1283-1290. Remnants of infectious microbes, including Staphylococcus, Streptococcus, Salmonella, Herpes Simplex, Escherichia coli, Chlamydia pneumoniae, Mycoplasma pneumoniae, Poryphromonas, spirochetes and other periodontal pathogens, Helicobacter pylori, and Archae are detected within arterial plaques in atherosclerosis and cerebral plaques in dementia by immunohistochemistry, electron microscopy, and hybridization of DNA oligonucleotides directed against microbial nucleic acids. Within infected arterial cells and brain cells the polyamines spermine and spermidine are synthesized by host cells from putrescine and the propylamino group of adenosyl methionine, causing depletion of cellular adenosyl methionine, decreased activation of cystathionine synthase, decreased biosynthesis of thioretinaco ozonide, and hyperhotnocysteinemia from dysregulation of methionine metabolism.

Bacteria that are demonstrated within arterial plaques occur as biofilms which are resistant to anti-microbial therapy, and dispersal of biofilms by free iron or epinephrine may contribute to plaque rupture, as reported by Lanter B B, Sauer K, Davies D O mBio 2014; 5: e01206-14. Biofilms, lipoprotein aggregates, and homocysteine contribute to rupture of vulnerable arterial plaques, as reported by Ravnskov U, McCully K S mBio 2014; 5: 301717-14. Proteolytic enzymes increase the susceptibility of bacterial biofilms to treatment with antibiotics by hydrolyzing the protein components of biofilm matrix containing extracellular polymeric substances that contribute to stability of biofilms, as reported by Selan L, Berlutti F, Passariello C, Comodi-Ballanti M R, Thaller M C Antimicrobial Agents and Chemotherapy 1993; 37: 2618-2621, and by Molobela I P, Cloete T E, Beukes M African Journal of Microbiology Research 2010; 4: 1515-1524. Glycosidic hydrolases, which hydrolzye the polysaccharide matrix of biofilms, are effective in dispersing biofilms of the alimentary pathogen, Pseudomonas aeruginosa, as reported by Baker P, Hill P J, Snarr B D et al Scientific Advances 2016; 2: e1501632. Furthermore, the powerful digestive enzymes of pancreatic extracts, including trypsin, chymotrypsin, deoxyribonuclease, ribonuclease, and amylase contribute to the dispersal of biofilms by hydrolyzing the proteins, nucleic acids, and polysaccharides of biofilm matrix within arterial plaques in atherosclerosis and within cerebral plaques in dementia.

The lipoproteins of plasma constitute an innate immune system that inactivates a wide variety of viruses, bacteria, protozoans, and other infectious microbes and their toxins by complexation and aggregation. Homocysteine thiolactone reacts with the free amino groups of the apoB protein of low-density lipoprotein to form aggregates that undergo spontaneous precipitation in vitro. Vulnerable plaques of arteries in atherosclerosis originate from obstruction of vasa vasorum of arterial wall by aggregates formed from lipoproteins complexed with microbial remnants, homocysteinylated lipoproteins, and lipoprotein autoantibodies in areas of high pressure, causing ischemia, degeneration of arterial wall cells and rupture into arterial intima to form a micro-abscess, the vulnerable plaque, as described by Ravnskov U, McCully K S Annals of Clinical and Laboratory Science 2009; 39: 3-16. The obstruction of vasa vasorum by lipoprotein aggregates is exacerbated by swelling and hyperplasia of endothelial cells, vasoconstriction, and fibrin deposition in the walls of arterioles. These changes in endothelial cell structure and function are manifestations of the endothelial dysfunction caused by hyperhomocysteinemia. Increasing evidence also implicates the presence of microbial remnants within the extracellular amyloid plaques and neurofibrillary tangles within neurons as factors in the pathogenesis of dementia and neurodegenerative diseases, as reported by McCully K S Journal of Alzheimer's Disease 2016; 45: 1283-1290.

Cultured fibroblasts from a child with homocystinuria caused by inherited deficiency of cystathionine synthase convert the sulfur atom of homocysteine to sulfate, demonstrating a pathway for conversion of homocysteine to sulfate without the intermediate formation of cystathionine. In this pathway thioretinamide is oxidized by superoxide to sulfite, alpha keto butyrate, ammonia and retinoic acid, catalyzed by ascorbic acid, and sulfite is oxidized to sulfate by sulfite oxidase. Sulfate is activated by reaction of sulfate and nicotinamide adenine dinucleotide to form adenosine phosphosulfate, catalyzed by electron transport from electron transport complexes, as reported by McCully K S Annals of Clinical and Laboratory Science 2016; 6: 435-438. Phosphoadenosine phosphosulfate is formed by phosphorylation of adenosine phosphosulfate by guanosine triphosphate (GTP), catalyzed by adenosine phosphosulfate kinase. Cell cultures derived from malignant tumors fail to oxidize homocysteine to sulfate, and the malignant phenotype is attributed to the resulting accumulation of intracellular homocysteine thiolactone. In normal cells and tissues phosphoadenosine phosphosulfate is utilized for sulfation of cholesterol, estrogens, steroid hormones, drug metabolites and glycosaminoglycans, such as chondroitin sulfate. In atherosclerosis, accumulation of mural sulfated glycosaminoglycans is an early manifestation of arterial plaque formation.

The failure of oxidation of homocysteine in liver of scorbutic guinea pigs and the pathway for conversion of homocysteine thiolactone to thioretinamide, sulfite, sulfate and phosphoadenosine phosphosulfate suggest participation of ascorbic acid in oxidative phosphorylation. Semidehydroascorbate is suggested as a product of oxidation of retinol to retinoic acid by superoxide and dehydroascorbate. Retinoic acid combines with homocysteine thiolactone to produce thioretinamide, catalyzed by cystathionine synthase, and thioretinamide combines with cobalamin to produce thioretinaco, aught in McCully U.S. Pat. No. 4,925,931. These observations suggest participation of electron transport in the activation of sulfate to adenosine phosphosulfate, catalyzed by the active site of oxidative phosphorylation, as reported by McCully K S Annals of Clinical and Laboratory Science 2016; 6: 435-438. Only higher eukaryotes contain cystathionine synthase with a heme oxygenase functional group, and the cystathionine synthase of prokaryotes contains no heme functional group. Since embryonic cells and malignant cells are deficient in the activity of cystathionine synthase, this formulation explains why malignant cells are deficient in oxidation of homocysteine thiolactone to sulfate because of deficiency of the heme oxygenase function of cystathionine synthase and why homocysteine thiolactone accumulates in malignant cells.

The enzyme cystathionase (cystathionine γ-lyase) is absent from human fetal liver, and the activities of cystathionine synthase and adenosyl methionine synthase are at reduced levels, compared with adult liver. Therefore, the trans-sulfuration pathway for conversion of homocysteine to cystathionine, cysteine and sulfate is inactive in fetal tissues, and the pathway for synthesis of sulfate from homocysteine thiolactone, involving synthesis of thioretinamide from retinol and homocysteine thiolactone and subsequent oxidation of thioretinamide to sulfite and sulfate by superoxide, is the source of glycosaminoglycan sulfate groups in fetal cells and tissues.

In the early 20^{th} century the embryologist John Beard discovered that trophoblastic cells of the embryo, which invade the uterine myometrium and endometrium during implantation of the fertilized embryo, are related to the asexual cycle of cellular organisms and are converted to placental cytotrophoblastic and syncytiotrophoblastic cells by the lytic action of enzymes produced by the pancreas of the developing fetus. Based on the concept that trophoblastic cells, which are distributed within developing tissues of the fetus, are similar in their cellular behavior to malignant cells, Beard introduced the enzyme treatment of cancer. This treatment consists of injection of enzymes and pro-enzymes extracted from porcine, bovine or Iamb pancreas into patients with various forms of primary or metastatic cancer. The trophoblastic theory of the origin of cancer is based on the assumption that cancer stem cells develop from the trophoblastic stem cells which migrate from the yolk sac of the developing embryo into somatic tissues, as described by Beard. The sensitivity of trophoblastic cells to oncolysis by pancreatic enzymes and pro-enzymes is related to the accumulation of homocysteinylated enzymes, plasma proteins, cellular proteins, DNA and RNA, and glycosaminoglycans, the amino groups of which are homocysteinylated by reaction with excess homocysteine thiolactone that accumulates within cells and tissues during aging, atherogenesis, carcinogenesis, and autoimmune diseases, as reported by McCully K S Annals of Clinical and Laboratory Science 1994; 24: 27-59. Evidence for the action of pancreatic enzymes and pro-enzymes in facilitating catabolism of homocysteinylated macromolecules in diseases of aging and increasing conversion of homocysteine to methionine by transmethylation is provided by the observation of elevated serum homocysteine in patients with cystic fibrosis and pancreatic insufficiency treated with pancreatic enzymes, compared with controls, as reported by Innis S M et al Journal of Pediatrics 2003; 143: 351-356.

In the early 20^{th} century the biochemist Otto Warburg discovered that embryonic tissues and malignant cells are unable to utilize oxygen for cellular metabolism but instead metabolize glucose to lactate as a source of cellular energy and ATP synthesis. Subsequent studies showed that carcinogenic chemicals decrease normal respiration by inhibition of oxygenases and by inhibition of transport of electrons by cytochrome enzyme systems. Taken together these early observations can be interpreted as examples of the clonal selection of malignant cells from trophoblastic stem cells that are deficient in the heme oxygenase activity of cystathionine synthase. The resulting failure of oxidation of retinol to retinoic acid and the failure of reaction of retinoic acid with homocysteine thiolactone to produce thioretinamide by these malignant cells will lead to deficient formation of thioretinaco and failure of oxidative phosphorylation and ATP synthesis, catalyzed by thioretinaco ozonide, as discussed by McCully K S Annals of Clinical and Laboratory Science 1994; 24: 27-59.

The failure of oxidative phosphorylation by malignant cell clones that are deficient in the heme oxygenase function of cystathionine synthase, resulting from decreased production of thioretinaco ozonide from cobalamin and thioretinamide, will lead to an embryonic form of oxidative metabolism in which ATP synthesis is dependent upon production of lactate from glucose, otherwise known as aerobic glycolysis. The growth of cultured human pancreatic adenocarcinoma cells in vitro is inhibited by thioretinamide or thioretinaco without cellular degeneration, but lactate production is increased, in contrast to normal fibroblasts, in which growth and lactate production are unaffected by thioretinamide or thioretinaco, as reported by McCully K S et al Research Communications in Chemical Pathology and Pharmacology 1992; 77: 125-128.

Hydrogen sulfide is a potent gaso-transmitter that senses oxygen concentrations in tissues, and the enzymes cystathionine synthase, cystathionase (cystathionine γ-lyase), and 3-mercaptopyruvate sulfotransferase produce hydrogen sulfide from cysteine, as reviewed by Polhemus D J et al Circulation Research 2014; 114: 730-737. Hydrogen sulfide decreases oxidative stress and counteracts experimental ischemia-reperfusion injury, hypertension, and renal failure. In an experimental mouse model hydrogen sulfide attenuates neurodegeneration and neurovascular dysfunction induced by intracerebral administration of homocysteine. Hydrogen sulfide was found to attenuate myocardial ischemia-reperfusion injury by preservation of oxygen consumption by mitochondria, as reported by Elrod J W et al Proceedings of the National Academy of Sciences USA 2007; 104: 15560-15565. The beneficial vascular effects of the allyl sulfide constituents of garlic, diallyl trisulfide and diallyl disulfide, are mediated by hydrogen sulfide, as reported by Benavides G A et al Proceedings of the National Academy of Sciences USA 2007; 104: 17977-17982.

The allyl sulfides, diallyl trisulfide and diallyl disulfide, are responsible for the homocysteine-lowering effects of aged garlic extract observed in folate-deficient rats by increasing adenosyl methionine in liver, which impairs the remethylation of homocysteine to methionine and enhances conversion of homocysteine to cystathionine by cystathionine synthase, as reported by Yeh Y Y et al The Journal of Nutrition 2006; 136: 745S-749S. In an animal model diallyl trisulfide protects against ethanol-induced oxidative stress and apoptosis by stimulating cystathionine synthase and cystathionase activities thereby increasing production of hydrogen sulfide by cystathionine y-lyase, as reported by Chen L Y et al International Immunopharmacology 2016; 36: 23-30. In diabetic rats diallyl trisulfide protects against the oxidative stress and apoptosis induced by hyperglycemia by stimulating production of hydrogen sulfide derived from cystathionine gamma-lyase, as reported by Tsai C Y et al International Journal of Cardiology 2013; 168: 1286-1297 and Tsai C Y et al International Journal of Cardiology 2015; 195: 300-310. These studies support the function of hydrogen sulfide in mediating the beneficial effects of diallyl trisulfide, diallyl disulfide, and other allyl sulfides on platelet aggregation, hypertension, elevated homocysteine, elevated cholesterol, oxidative stress, and apoptosis occurring in cardiovascular disease. Diallyl trisulfide is the most potent diallyl sulfide compound in providing beneficial effects, compared with diallyl disulfide and diallyl sulfide, which are less potent. The unsaturated allyl groups of these polysulfide compounds facilitate their entry through plasma membranes into cells, since the corresponding saturated propyl derivatives have no beneficial cardiovascular effects.

The allyl sulfides diallyl trisulfide and diallyl disulfide not only have beneficial cardioprotective effects, but many studies document potent anti-neoplastic and anti-carcinogenic effects of these compounds. Treatment of cultured human glioblastoma cells with allyl sulfides causes production of reactive oxygen species and induction of apoptosis by activation of the JNK-1 pathway, as reported by Das A et al Cancer 2007; 110: 1083-1094. In this study increases in intracellular free calcium [Ca²⁺], expression of calreticulum and activation of caspase-4 indicate involvement of endoplasmic reticulum stress and decreased mitochondrial membrane potential in induction of apoptosis, resulting in reduced cell viability. The potential efficacy of diallyl trisulfide in treatment of human glioblastoma is attributed to the ability of this compound to penetrate the blood brain barrier, increasing its concentration within glioblastoma cells.

Diallyl trisulfide induces apoptosis in cultured human breast cancer cells through activation of c-jun N-terminal kinase (JNK) and activator protein-1 (AP-1), as reported by Na H K et al Biochemical Pharmacology 2012; 84: 124101250. Diallyl trisulfide causes apoptosis and reduced cell viability in cultured human breast cancer cells and transgenic mouse breast cancer cells, whereas cultured normal breast ductal cells were resistant to growth inhibition and induction of apoptosis by dially trisulfide, as reported by Chandra-Kuntal K et al Breast Cancer Research Treatment 2013; 138: 69-79. Human breast cancer cells in culture which are exposed to diallyl trisulfide exhibit suppression of matrix metalloproteinase 2/9 (MMP-2/9) by blocking the nuclear factor kappa B (NFκB) and ERK/MAPK signaling pathways, potentially inhibiting migration and invasion of metastatic breast cancer, as reported by Liu Yet al PLOS one 2015; 10: e0123781. Another study demonstrates targeting and growth inhibition of breast cancer stem cells by diallyl trisulfide through inhibition of forkhead box Q1 (FoxQ1), as reported by Kim S H et al Journal of Biological Chemistry 2016; 291; 13495-13508.

In studies of human skin cancer diallyl trisulfide was demonstrated to inhibit growth of human melanoma cells and basal cell carcinoma cells in culture by increasing the levels of intracellular reactive oxygen species, leading to DNA damage, and by inducing G2/M mitotic arrest, endoplasmic reticulum stress, and mitochondria-mediated apoptosis, including the caspase-dependent and caspase-independent pathways, as reported by Wang H C et al Annals of the New York Academy of Sciences 2012; 1271: 44-52.

In a study of cultured human pancreatic cancer cells, diallyl trisulfide was demonstrated to produce apoptosis and suppression of growth, but non-tumorigenic pancreatic ductal epithelial cells were significantly more resistant to suppression of growth and induction of apoptosis by diallyl trisulfide, compared with pancreatic cancer cells, as reported by Ma H B et al World Journal of Gastroenterology 2014; 20: 193-203. Pancreatic cancer evolves from dysplastic epithelial precursor cells, termed pancreatic intra-epithelial neoplasia (PanIN). Different degrees of dysplasia are classified as PanIN-1, PanIN-2, and PanIN-3, regarding progression of the dysplastic changes to malignant and invasive pancreatic adenocarcinoma cells. Diallyl sulfides, including diallyl trisulfide, are potentially effective in preventing progression of PanIN cells to the malignant phenotype, delaying the onset of invasive pancreatic cancer.

In a study of human promyelocytic leukemia cells in culture, garlic oil and onion oil were demonstrated to inhibit cellular proliferation and induce differentiation, an effect that was similar to the effect of retinoic acid on differentiation, as reported by Seki T et al Cancer Letters 2000; 160: 29-35. There was no cellular toxicity of garlic oil, as assayed by release of lactate dehydrogenase from cells, and the analysis of garlic oil by gas chromatography-mass spectroscopy revealed diallyl disulfide 56.6%, methyl allyl disulfide 12.0%, diallyl trisulfide 11.8%, diallyl sulfide 1.94% and dimethyl trisulfide 1.4%. The toxic effects of retinoic acid therapy are obviated by use of thioretinamide, which is unassociated with toxicity.

The proliferation of cultured human squamous cell carcinoma cells and immortalized human oral keratinocytes is inhibited by highly purified inorganic sulfur by increasing cellular apoptosis, as reported by Lee J et al Toxicology in Vitro 2008; 22: 87-95. Highly purified inorganic sulfur reduces the motility and invasion of human breast cancer cells as assessed by in vitro culture assays, as reported by Kim J J et al Nutrition Research and Practice 2011; 5: 375-380. These studies document highly purified inorganic sulfur as 99.9% pure, but no information is presented regarding the molecular forms of sulfur, regarding orthorhombic sulfur, which is characterized by S₈, a cyclic octa-sulfur species, monoclinic sulfur, which is a cyclic octa-species of sulfur with different molecular packing, other cyclosulfurs containing 7 to 12 sulfur atoms per ring, or catena-sulfur, which contains chains of sulfur atoms of different lengths. Hydrogen sulfide is partly un-dissociated and undergoes partial dissociation into hydrosulfide anion (HS) and sulfide anion (S²⁻) at physiological pH and body temperature, and sulfide anion is suggested as the active sulfur species that mediates the growth inhibition and apoptotic effects of sulfur and hydrogen sulfide by modulating the activity of cytochrome C and caspase-3, both of which are essential to hyperhomocysteinemia-induced oxidative reactions, as reviewed by Ingenbleek et al Nutrition Reviews 2013; 71: 413-432.

Exposure of the experimental nematode, Caenorhabtidis elegans, to diallyl trisulfide increases its lifespan by approximately 12% by activation of the nuclear factor skn-1, as reported by Powolny A A et al Experimental Gerontology 2011; 46: 441-452, The nuclear factor An-1 is expressed in intestinal cells and neurons in nematodes and is the homologue of the mammalian Nuclear Factor Erythroid-derived 2-Related Factor (Nrf2). Skn-1 coordinates the responses to oxidative stress, and a similar requirement for skn-1 was found for the increased lifespan produced by metformin or dietary restriction in C. elegans. The oxidative stress and hyperhomocysteinemia associated with aging are attributable to cellular deficiency of thioretinaco ozonide and the resulting decrease in oxidative phosphorylation observed in aging, as reported by McCully K S Annals of Clinical and Laboratory Science, 2015; 45: 222-225.

Although many of the organic ally! sulfides and other organosulfur compounds of garlic have anti-thrombotic and anti-cancer effects, the most potent of these compounds is diallyl trisulfide, the inhibitory concentration (IC₅₀) of which against proliferation of human colon carcinoma cells in vitro is 5.0 μM, as reported by Ariga T et al Biofactors 2006; 26: 93-103. Moreover, diallyl trisulfide and other organosulfur compounds of garlic have potent anti-bacterial, anti-fungal, and anti-animal effects, facilitating the beneficial effects against atherosclerosis and Alzheimer's dementia, the pathogenesis of which is promoted by infectious microbes, as reported by Ravnskov U et al American Journal of the Medical Sciences 2012; 344: 391-394 and McCully K S Journal of Alzheimer's Disease 2016; 54: 1283-1290. The allyl sulfide components of aged garlic extract enhance innate immunity and inhibit proliferation of viral, fungal, parasitic, protozoan and bacterial infectious agents, including Salmonella, Listeria, Escherichia coil, Helicobacter pylori, Mycobacterium tuberculosis, bacterial biofilm pathogens, rhinovirus, cytomegalovirus, Herpes simplex, influenza virus, Candida albicans, Aspergillus flavus, Cryptosporidium, Toxoplasma, Giardia, and Plasmodium. Aged garlic extract modulates innate immunity by increasing the activity of macrophages and natural killer cells and by increasing production of T and B cells. Clinical trials have documented the ability of aged garlic extract to decrease the number, severity and duration of upper respiratory infections, as reviewed by Reid K The Journal of Nutrition 2016; 146: 389S-396S.

In a study of nitric oxide (NO) recruitment of monocytes in mice with femoral artery ligation, absence of cystathionine γ-lyase in knockout mice failed to produce NO, but wild type mice with normal cystathionine γ-lyase activity were associated with increased NO production, hydrogen sulfide production and monocyte recruitment in ischemic tissues, as reported by Koltun G K et al Cardiovascular Research 2015; 107: 590-600. Treatment of the cystathionine γ-lyase-deficient knockout mice with dially trisulfide restored ischemic vascular remodeling, monocyte recruitment, and cytokine expression by increasing hydrogen sulfide production and restoring nitric oxide bioavailability. NO has powerful anti-microbial activity because of formation of peroxynitrite (OONOO⁻) from superoxide (O₂⁻). Large quantities of NO are produced during infections caused by a variety of pathogens, and peroxynitrite has potent microbiocidal activity by induction of nitrative stress. Both reactive oxygen species and reactive nitrogen species are delivered to phagosomes of neutrophils and macrophages to mediate anti-microbial activity, as discussed by McCully K S Journal of Alzheimer's Disease 2016; 54: 1283-1290.

Diallyl disulfide increases mRNA synthesis by the heme oxygenase gene, HMOX1, and heme oxygenase is up-regulated in cultured liver cells exposed to ethanol, as reported by Charron C S et al The Journal of Nutrition 2016; 146: 444S-449S. Diallyl disulfide also suppresses lactate dehydrogenase and aspartate transaminase activities, suppresses malondialdehyde concentrations, and increases glutathione concentrations, contributing to protection against ethanol-induced injury to hepatic cells by up-regulation of HHMOX1. Thus allyl sulfides have the capacity to enhance biosynthesis of thioretinamide from retinol and homocysteine thiolactone by oxidizing retinol to retinoic acid through up-regulation of the heme oxygenase function of cystathionine synthase, thereby increasing formation of thioretinaco ozonide, adenosyl methionine, and facilitating oxidative phosphorylation in normal and regenerative cells. Malignant cells, which are deficient in the heme oxygenase function of cystathionine synthase, do not form thioretinamide and thioretinaco ozonide, but the growth of malignant cells is inhibited by the pro-apoptotic effects of allyl sulfides and ozone.

In screening for synthetic furanonaphthoquinone compounds with cytotoxic activity towards various leukemia cells and multiple myeloma cells, Desmond J C et al British Journal of Haematology 2005; 131: 520-529 demonstrated that 2-methyl-naphtho[2,3-b]furan-4,9 dione (FNQ3) decreases the growth and increases apoptosis within cultured human leukemia and myeloma cell lines. Normal control cell lines are resistant to the cytotoxic effects of FHQ3, requiring a 10-fold increase in concentration to produce apoptosis and growth inhibition. The cytotoxic effect of FNQ3 is mediated by mitochondrial collapse, as measured by depolarization of the mitochondrial membrane, resulting in appearance of a sub-G1 (apoptotic) population of cultured cells. Moreover, FNQ3 markedly enhances granulocytic differentiation of human myeloid leukemia cells in the presence of low concentrations of retinoic acid or dihydroxy vitamin D3. The mitochondrial dysfunction induced by FNQ3 can be interpreted as competitive inhibition of electron transfer from respiratory complexes to the active site of oxidative phosphorylation, as mediated by coenzyme Q-10, resulting in a decreased proton flow to F1F0 complexes and the resulting decreased membrane pate tial, as discussed by McCully K S Annals of Clinical and Laboratory Science; 2015: 222-225.

Multiple naphthoquinones isolated from the bark and roots of an African shrub, Newbouldia laevis, used in traditional medicine were demonstrated to have prominent antifungal and antibacterial properties, as reported by Gafner S, et al Phytochemistry 1996; 42: 1315-1320. Additional furanonaphthoquinones, atraric acid and a benzofuran were also isolated from the stem barks of Newbouldia laevis, as reported by Gormann R, et al Phytochemistry 2003; 64: 583-587. In a similar study of the roots of the “roble real” tree of Puerto Rico and the Dominican Republic, Ekmanianthe longiflora, multiple furanonapthoquinones with anticancer and antimicrobial activity were found to have cytotoxic effects in cultured human breast and lung cancer cell lines, as reported by Peraza-Sanchez S R, et al Journal of Natural Products 2000; 63: 492-495. Studies of naphthoquinones and analogues from Avicennia (mangrove) trees disclosed inhibitory effects against mouse skin tumor formation in carcinogenesis testing, as reported by Itoigawa M, et al Cancer Letters 2001; 174: 135-139. Synthetic derivatives of furanonaphthoquinones isolated from plants were demonstrated to have cytotoxic activity against human tumor cells incubated against KB cells, as reported by Ogawa M, et al Bioscience Biotechnology and Biochemistry 2006; 70: 1009-1012. The basis for toxicity was considered by the authors to be related to intercalation within DNA and free radicals. Two newly discovered cytotoxic naphthoquinones were isolated from roots and stems of the Madagascar plant, Mendoncia cowanii (vahimpianaomby), and found to be cytotoxic for human ovarian cancer cell lines, as reported by Williams R B, et al Planta Medica 2006; 72: 564-566. All of these furanonaphthoquinones with cytotoxic activity potentially affect mitochondrial function through inhibition of electron transfer, as mediated by coenzyme Q-10, from respiratory complexes to the active site of oxidative phosphorylation. Malignant cells are more susceptible to the cytotoxic effects of these furanonaphthoquinones because of the lower concentration of thioretinaco ozonide within the mitochondrial membranes of malignant cells, compared with normal cells.

The signal transducer and activation of transcription 3 (Stat3) is frequently detected in breast cancer cell lines but not in normal breast epithelial cells, and a virtual database screening protocol disclosed a natural product molecule, deoxytetrangomycin, an angucycline antibiotic (National Cancer Institute 628869) with potent inhibitory activity against Stat3 activity in human breast cancer cell lines, as reported by Song H et al Proceedings of the National Academy of Sciences USA 2005; 102: 4700-4705. A systematic study of naphthoquinone derivatives with inhibitory activity against Stat3 signaling in cancer stem cells disclosed a novel class of furanonaphthoquinone molecules with inhibitory activity against cancer stem cell proliferation, as taught in Jiang et al U.S. Pat. No. 8,877,803. The most active of these compounds is napabucasin, 2-carboxymethyl-naphtho[2,3-b]furan-4,9-dione, which inhibits gene transcription dependent upon Stat3, thereby suppressing gene expression of cancer sternness, blocking spherogenesis of cultured cancer cells, and causing apoptosis of a wide variety of cancer cell types, as reported by Li Y et al Proceedings of the National Academy of Sciences USA 2015; 112: 1839-1844.

In a study of human prostate cancer cell cultures, napabucasin was demonstrated to increase apoptosis, inhibit cell proliferation, cell motility, cell survival, and colony formation ability of prostate cancer stem cells, as reported by Zhang Y et al Cancer Medicine 2016; 5: 1251-1258. Moreover, an in vivo study demonstrated that napabucasin inhibits growth of prostate cancer xenografts from these cancer cell cultures in athymic mice.

Diallyl trisulfide, the cancer chemopreventive constituent of garlic, inhibits phosphorylation of Stat3 in prostate cancer cells in culture and in vivo, as reported by Chandra-Kuntal K and Singh S V Cancer Prevention Research (Phila) 2010; 3: 1473-1483. In this study diallyl trisulfide inhibited nuclear translocation of Stat3 and dimerization of Stat3 in prostate cancer cell lines. In addition, diallyl trisulfide inhibited prostate cancer development in a transgenic mouse model, correlating with a decrease in phosphorylated Stat3. Diallyl trisulfide also inhibited migration of prostate cancer cells, an indicator of metastatic potential, in an in vitro membrane migration assay. In a study of mouse colitis induced by dextran sulfate, diallyl trisulfide suppressed Stat3 and NF-κB expression, resulting in decreased inflammation of the colon, as reported by Lee H J et al Biochemical and Biophysical Research Communications 2013; 437: 267-273.

High concentrations (300 mM) of methylsulfonylmethane (dimethyl sulfone), a non-toxic volatile dietary constituent, suppress growth of cultured human breast cancer cell lines by decreasing the phosphorylation of Stat3 and inhibiting binding of Stat3 and Stat5 to DNA, as reported by Lim E J et al PLoS One 2012; 7: e33361. In this study methylsulfonylmethane reduced viability and induced apoptosis of human breast cancer cell lines and inhibited migration of cultured metastatic human breast carcinoma cells, as shown by in vitro icroscopy. In addition, methylsulfonylmethane inhibited growth of human breast carcinoma xenografts in athymic mice by reduction of insulin-like growth factor (IGF-1) and down-regulation of vascular endothelial growth factor (VEGF) and Stat3. In hormone responsive breast cancer cells, methylsulfonylmethane had the ability to down-regulate the expression of triple-negative hormone receptors.

In a study of bone osteoclast formation, methylsulfonylmethane was demonstrated to inhibit osteoclastogenesis induced by receptor activator NF-κB ligand (RANKL) in bone marrow macrophages by suppression of NF-κB and Stat3 activities, as reported by Joung J H et al PLoS ONE 2016; 11: e0159891. Thus methylsulfonylmethane has the potential to attenuate RANKL-induced osteoclastogenesis by down-regulation of both NF-κB and Stat3, suggesting methylsulfonylmethane as a potential therapeutic modality for treatment of disorders characterized by bone loss.

ONA, 3,4-dimethyl-5-(1E-propenyl)-tetrahydrothiophen-2-sulfoxide-S-oxide, is a stable, sulfur-containing compound isolated from onions which has a marked inhibitory effect on production of the M2 macrophage phenotype, as reported by Fujiwara Y et al Molecular and Nutritional Food Research 2016; 60: 2467-2480. ONA inhibits Stat3 activation, causing inhibition of the immunosuppressive activity of myeloid suppressor cells and inhibition of tumor growth of a human glioblastoma cell line, a human osteosarcoma cell line, and a mouse osteosarcoma cell line. Furthermore, ONA suppressed both subcutaneous tumor development and lung metastases in a mouse tumor model and in a xenograft of mouse osteosarcoma cells in athymic mice.

Screening of chemical libraries led to the identification of stattic, a non-peptide molecule which selectively inhibits activation, dimerization, and nuclear translocation of Stat3 and increases the apoptotic rate of Stat3-dependent breast cancer cell lines, as reported by Schust J et al Chemistry & Biology 2006; 13: 1235-1242. The stattic molecule, 6-nitro-benzo[b]thiophene-1,1-dioxide, binds a phosphotyrosine-containing peptide derived from the gp130 receptor to the Stat3 SH2 domain in a temperature-dependent manner, resulting in inhibition of Stat3 activity. Substitution of an amine group for the nitro group, elimination of the nitro group, or hydrogenation of the thiophene group caused inactivation of the stattic molecule for inhibition of Stat3.

In a study of acute lung injury induced by limb ischemia/reperfusion in rats, administration of sulfur dioxide, SO₂, from Na₂SO₃/NaHSO₃ was found to attenuate acute lung damage and inflammation, as reported by Zhao Y R et al Journal of Physiological Science 2016; 66: 229-239. SO₂ decreased expression of lung phosphorylated Stat3 induced by limb ischemia/reperfusion injury. However, SO₂ had no effect on expression of lung Akt and p38 signaling pathways. SO₂ is considered to be a candidate messenger mediator of inflammatory and anti-inflammatory cytokines in plasma during acute lung injury induced by limb ischemia/reperfusion.

A series of 8 thiosulfonate drug hybrids was synthesized and tested for binding to the Stat3-SH2 domain, as reported by Gabriele E et al Journal of Enzyme Inhibition and Medicinal Chemistry 2017; 32: 337-344. These drug hybrids are derivatives of methylsulfonylmethane, which is an inhibitor of Stat3 signaling. Assay for inhibition of proliferation of a colorectal adenocarcinoma cell line revealed moderate activity of several of these hybrid compounds, but other compounds were inactive in the assay system. The hybrid, S((methylsulfonyl)methyl) 4-amino-2-hydroxybenzothioate, was considered worthy of further investigation because of its moderate inhibition of Stat3 signaling and cytotoxic activity against proliferation of the cancer cell line.

A hybrid molecule, tacrine-8-hydroxyquinoline, was evaluated for ability to inhibit Aβ amyloid deposition and inhibition of neurodegeneration in two experimental models, as reported by Antequera D et al Neurobiology of Disease 2012; 46: 682-691 . This compound was found to promote degradation of intracellular Aβ in astrocytes and to protect against Aβ toxicity in cultured astrocytes and neurons, but no study of Stat3 signaling was reported.

A study of tacrine-quinone hybrid molecules revealed antioxidant activity in cultured mouse neurons, ability to counteract the toxicity of Aβ, provide anti-cholinesterase activity, and ability to cross the blood brain barrier, as reported by Nepovimova E et al Journal of Medicinal Chemistry 2014; 57: 8576-8589. No study of the effect of this molecule on Stat3 signaling was reported. The naphthoquinone, 2-methoxystypandrone, was found to improve mouse brain damage induced by ischemia/reperfusion injury, reduce production of free radical oxygen species, and preserve blood brain barrier integrity by reduction of the NF-κB signaling pathway, as reported by Chem C M et al Biochemical Pharmacology 2014; 87: 502-514, No study of the effect of this molecule on Stat3 signaling was reported. The compound also promotes neurodevelopmental protein expression and endogenous neurogenesis by inactivation of glycogen synthase kinase 3 (GSK3) and up-regulation of doublecortin, a marker of neuroblast proliferation, β-catenin and Bcl-2. The authors consider these activities in animal models worthy of study in human trials.

Tryphostin B42 (AG-490) is an inhibitor of EGFR and JAK2 signaling that induces apoptosis in leukemia cells, and a combination of methylsulfonylmethane with tyrphostin B42 suppresses bladder tumor growth in human bladder cancer lines and xenografts in athymic mice, as reported by Joung Y H et al International Journal of Oncology 2014; 44: 883495. The combination therapy reduced signaling molecules, including Stat3, Stat5b, IGF-1R, VEGF and VEGF-R2, which are involved in proliferation, invasion and metastasis of human bladder cancer. In the xenograft experiments the combination of tyrphostin B42 and methylsulfonylmethane significantly suppressed angiogenesis and metastasis to lung.

The various compositions under study for the treatment of cancer do not include combinations of thioretinamide with diallyl trisulfide or furanonaphthoquinones or the abnormal accumulation of homocysteine in cells. The various compositions have not combined thioretinamide with other compositions designed to work with the immune system to treat cancer. The prior art has not combined thioretinamide with other compositions which pass through the blood brain barrier to promote the apoptosis of malignant cells and inhibit the apoptosis of normal cells.

### SUMMARY OF THE INVENTION

My invention includes novel therapeutic compositions of matter for stimulating the immune system and increasing the apoptosis of malignant cells while preventing apoptosis in normal cells. The novel compositions are also capable of crossing the blood brain barrier to treat glioblastoma. The novel compositions include thioretinamide along with highly purified inorganic sulfur or organic allyl sulfides or furanonaphthoquinones alone or optionally with pancreatin or cobalamin. The highly purified inorganic sulfur compounds are cyclic sulfur compounds having 6 to 12 atoms per ring such as S₈, a cyclic octasulfur compound. The organic allyl sulfide is preferably diallyl trisulfide and is of purity greater than 10%. The furanonaphthoquinone is preferably napabucasin and is of purity greater than 10%.

The purity of the organic allyl sulfides or furanonaphthoquinones is related to the strength of the immune system and the type of apoptosis required for prevention or treatment of malignant cells. The novel formulations of thioretinamide along with highly purified inorganic sulfur or organic allyl sulfides or furanonaphthoquinone or optionally with pancreatin or cobalamin may be combined with retinol, adenosyl methionine, folate or pyridoxal to enhance the activity of the cystathionine synthase and methionine synthase of target cells.

The method of my invention consists of administration of thioretinamide by oral, parenteral, or intravenous routes of delivery, combined with highly purified inorganic sulfur, allyl sulfides, and furanonaphthoquinones along optionally with pancreatic enzymes and pro-enzymes to degrade homocysteinylated macromolecules, the vitamins folate, pyridoxal, and cobalamin to enhance the activity of cystathionine synthase and methionine synthase, adenosyl methionine to activate cystathionine synthase by allosteric effects, essential amino acids including tryptophan to enhance endogenous transthyretin formation, ascorbate with mixed bioflavonoids to catalyze oxidation of retinol to retinoic acid, nitrilosides such as amygdalin to provide cyanide for combination with hydrogen sulfide, and allyl sulfides, including diallyl trisulfide and diallyl disulfide, to up-regulate the activities of cystathionine synthase, cystathionase and heme oxygenase for production of hydrogen sulfide and furanonaphthoquinones, including napabucasin, in order to promote apoptosis in malignant cells and to prevent apoptosis in normal cells for the purpose of preventing and treatment of diseases of aging.

My invention also encompasses administration of retinol by oral, parenteral, or intravenous routes of administration as a metabolic precursor of retinoic acid, in combination with pancreatic enzymes and pro-enzymes, the vitamins folate, pyridoxal and cobalamin, adenosyl methionine, essential amino acids including tryptophan, ascorbate with mixed bioflavonoids and nitrilosides in order to facilitate catabolism of homocysteinylated macromolecules and enhance endogenous biosynthesis of thioretinamide and thioretinaco, allyl sulfides including diallyl trisulfide and diallyl disulfide to enhance hydrogen sulfide synthesis by cystathionine synthase and cystathionase, and furanonaphthoquinones including napabucasin and allyl sulfides including allyl trisulfide to inhibit Stat3 signaling to promote apoptosis of cancer stem cells and to prevent apoptosis of normal cells for the purpose of preventing and treatment of cancer diseases the incidence of which increases with aging and increased levels of homocysteine. This metabolic protocol of thioretinamide administration with pancreatic enzymes and allyl sulfides will decrease the accumulation of homocysteinylated macromolecules and enhance endogenous hydrogen sulfide biosynthesis for the purpose of decreasing oxidative stress induced by homocysteine, enhancing oxidative metabolism and apoptosis of malignant cells and preventing apoptosis of normal cells occurring in diseases of aging. In diseases exacerbated by a microbial etiology, such as atherosclerosis, cancer or dementia, use of allyl sulfides, proteolytic enzymes, and furanonaphthoquinones together with appropriate antibiotics and antimicrobial triglycerides counteracts microbial growth and inflammation, facilitating the resolution of pathological lesions by the catabolism of homocysteinylated macromolecules, inhibition of microbial growth, and regeneration of normal cells in tissues affected by diseases of aging.

My invention is useful in promoting antigen presentation by dendritic cells in the activation of natural killer cells, which cause apoptosis of malignant cells by production of ozone and other oxygen radicals, activated by singlet oxygen and antibodies to neoplastic antigens in immunotherapy of cancer. My invention is also useful in preventing the progression of dysplastic cells to the malignant phenotype, thereby preventing cancer development from the effects of oncogenic microbes, including oncogenic viruses, carcinogenic chemicals, ionizing radiation or ultra-violet radiation, and oncogenic genetic alleles, in production of malignant cells that are deficient in the heme oxygenase function of cystathionine synthase and deficient in thioretinaco ozonide.

### DETAILED DESCRIPTION OF THE INVENTION INCLUDING BEST MODE

My invention relates to a method for metabolic control of utilization of thioretinamide in prevention and treatment of cancer, and degenerative diseases associated with an immature or reduced functioning immune system due to environment or aging. These diseases are all characterized by an abnormality of methionine metabolism in which an increased concentration of homocysteine is demonstrated by assaying plasma or other body fluids for homocysteine bound to proteins by disulfide bonds. The abnormality of methionine metabolism in these diseases is caused by loss or depletion of thioretinamide from the cells of the body during aging and disease such as cancer. Inadequate oxidation of the retinol which is transported to cells and tissues of the body by retinol binding protein and transthyretin, caused by the superoxide produced by the heme oxygenase function of cystathionine synthase, leads to decreased endogenous synthesis of thioretinamide from retinoic acid and homocysteine thiolactone. The resulting decrease in concentration of cellular thioretinamide leads to decreased production of thioretinaco and thioretinaco ozonide from thioretinamide, cobalamin and ozone. As a result, oxidative phosphorylation is inhibited because of cellular deficiency of thioretinaco ozonide, leading to accumulation of toxic free radical compounds and producing oxidative stress. In addition, the decreased biosynthesis of thioretinamide leads to increased production of homocysteine thiolactone from methionine and increased homocysteinylation of the free amino groups of proteins, deoxyribonucleic acids, ribonucleic acids, glycosaminoglycans and other macromolecules containing free amino groups by excess homocysteine thiolactone, impairing cellular function and causing accelerated aging of cells and tissues, contributing to the pathogenesis of degenerative diseases of aging such as cancer. In the embodiment of my invention without pancreatic enzymes for catabolism of homocysteinylated macromolecules, the use of thioretinamide with allyl sulfides, including diallyl trisulfide, and furanonaphthoquinones, including napabucasin, together with vitamins of the homocysteine-lowering protocol, provide beneficial effects on prevention and therapy of cancer and other diseases of aging, because of the potent apoptotic effects of allyl sulfides and furanonaphthoquinones and the effects of thioretinamide on production of thioretinaco ozonide in aging cells.

My invention overcomes the ineffective metabolic regulation of oxidative stress in human disease of the prior art by a novel method and compounds for the enhancement of endogenous biosynthesis of thioretinamide and thioretinaco within cells and tissues, thereby stimulating cellular oxidative metabolism and reducing the endogenous accumulation of reactive oxygen species and reducing the degradation of cellular and tissue constituents by free radical substances in chronic degenerative diseases. In addition, my invention overcomes the overproduction of homocysteine thiolactone by increasing its conversion to cysteine, metabolites of cysteine, and sulfate, preventing the deleterious homocysteinylation of macromolecules that is characteristic of degenerative diseases of aging such as cancer. My invention decreases the concentration of homocysteinylated macromolecules, such as homocysteinylated deoxyribonucleic acid and ribonucleic acid, homocysteinylated enzymes and other proteins, and homocysteinylated glycosaminoglycans by supplying exogenous digestive enzymes derived from pancreas, which release free homocysteine from homocysteinylated macromolecules, thereby increasing catabolism d excretion of homocysteine metabolites.

My invention also increases the endogenous production of hydrogen sulfide by increasing the activities of cystathionine synthase and cystathionase by supplying allyl trisulfide and other active allyl sulfides, thereby decreasing oxidative stress induced by homocysteine, enhancing oxidative metabolism, promoting apoptosis of malignant cells and preventing apoptosis of normal cells occurring in cancer. My invention utilizes allyl trisulfide and other allyl sulfides and napabucasin and other furanonaphthoquinones, which have the capacity to cross the blood brain barrier for the treatment of cerebral neoplasms and to cross membranes of malignant cells in treatment of neoplasms of other organs of the body. My invention also inhibits progression of atherosclerosis and dementia by providing allyl trisulfide and other active allyl sulfides and by providing napabucasin and other active furanonaphthoquinones, which have potent anti-microbial activity, thereby preventing progression of microbial infections that are characteristic of these diseases. By proteolysis of extracellular matrix polymers of biofilms, my invention also increases the susceptibility of biofilms to dispersal and inhibition by antibiotics, allyl sulfides, furanonaphthoquinones and medium chain saturated mono-glycerides with anti-microbial activity. At the same time my invention enhances innate immunity by providing increased intracellular thioretinaco, which has the capacity to bind ozone to form thioretinaco ozonide, thereby preventing damage to regenerative normal cells which utilize thioretinaco ozonide for oxidative metabolism. My invention is also useful in promoting presentation of antigens of malignant cells to dendritic cells for the activation of natural killer cells, which cause apoptosis of malignant cells by production of ozone and other oxygen radicals, activated by singlet oxygen and antibodies that are useful in the immunotherapy of cancer.

My invention works with and strengthens the immune system while inducing the apoptosis of cancer cells while protecting normal cell function and ameliorates the course of cancer by preventing accumulation of homocysteine within affected cells, preventing oxidative stress from free radical accumulation within cells and tissues, preventing accumulation of homocysteinylated macromolecules with impaired function, increasing endogenous production of hydrogen sulfide from homocysteine by the action of cystathionine synthase and cystathionase, thereby increasing apopotosis of malignant cells and decreasing apoptosis of normal cells in the treatment of cancer,

TABLE 1
Plasma homocysteine levels and risk of diseases of aging

| | | | Plasma homocysteine |
|---|---|---|---|
| Disease risk | Gender | Age | (μmol/L) |
| Low | Male | 20-40 | 4-8 |
| Low | Female | 20-50 | 4-8 |
| Mild | Male | 40-60 | 8-12 |
| Mild | Female | 50-60 | 8-12 |
| Moderate | Male | 50-70 | 10-14 |
| Moderate | Female | 60-70 | 10-14 |
| High | Male | 60-80 | 12-20 |
| High | Female | 70-80 | 12-20 |
| Very high | Male | 60-90 | 16-30 |
| Very high | Female | 70-90 | 16-30 |

As demonstrated in Table 1, the risk of diseases of aging, such as cancer increases with increasing plasma homocysteine levels. Risk increases at an earlier age for males, compared with females. After menopause, however, risk increases in females to attain a similar disease risk, compared with males of the same age. In Example 1, a 75 year old man with stage IV prostate cancer had a plasma homocysteine level of 14.0 μmol/L, corresponding to moderate to high disease risk; after therapy with the metabolic protocol for two years, the homocysteine level was 9.5 μmol/L. In Example 2, a 75 year old woman with macular degeneration and cognitive impairment had a plasma homocysteine level of 15.4 μmol/L, corresponding to high disease risk; after therapy with the metabolic protocol for 2 years, the homocysteine level was 8.5 μmol/L. In Example 3, a 60 year old man with acute coronary syndrome had a plasma homocysteine level of 15.8 μmol/L, corresponding to high disease risk; after therapy with the metabolic protocol for two years, the homocysteine level was 10.5 μmol/L. In Example 4, a 65 year old male with metabolic syndrome and early renal failure had a plasma homocysteine level of 16.5 μmol/L, corresponding to very high disease risk; after therapy with the metabolic protocol for two years, the homocysteine level was 10.5 μmol/L. In Example 5, a 70 year old man with arteriosclerosis and aortic aneurysm had a plasma homocysteine level of 18.5 μmol/L, corresponding to very high disease risk; after therapy with the metabolic protocol for two years, the homocysteine level was 10.2 μmol/L. In Example 6, a 65 year old woman with stroke had a plasma homocysteine level of 18.0 μmol/L, corresponding to very high disease risk; after therapy with the metabolic protocol for two years, the homocysteine level was 10.5 μmol/L. In Example 7, the addition of thioretinaco or thioretinamide and diallyl trisulfide or napabucasin to the culture medium of cultured human adenocarcinoma cells reduced cellular viability to 6.7% to 9.5%, compared with control cultures without added thioretinamide, diallyl trisulfide, or napabucasin (Table 2).

Thioretinamide (TR), also known as N-homocysteine thiolactonyl retinamide (NHTR), can be prepared, as described in U.S. Pat. Nos. 4,618,685, 6,054,595, and 6,287,818 by the reaction of homocysteine thiolactone with retinoic acid. Thioretinaco (TR₂Co), also known as N-homocysteine thiolactonyl retinamido cobalamin ((HTHR)₂Cbl), can be prepared, as described in U.S. Pat. No. 4.925,931, by reaction of thiroetinamide with 5′-deoxyadenosyl cobalamin (Cbl). Thioretinaco ozonide (TR₂CoO₃), can be prepared, as described in U.S. Pat. No. 5,565,558 by reaction of ozone with thioretinaco. Preservation of cellular thioretinaco ozonide by membranergic proteins and by a liposomal complex of ATP and oxygen with thioretinaco ozonide prolongs survival and counteracts the aging process, as taught in U.S. Pat. Nos. 5,565,558 and 6,696,082.

The specific and essential components of my invention comprise a metabolic protocol delivered by oral, intravenous or parenteral routes of administration. The protocol consists of (1) synthetic thioretinamide or dietary retinol as a precursor of endogenous thioretinamide biosynthesis; (2) ascorbate with bioflavonoids to promote oxidation of retinol to retinoic acid by dehydroascorbate and biosynthesis of thioretinamide from homocysteine thiolactone, catalyzed by the herne oxygenase function of cystathioinine synthase; (3) cobalamin as a precursor of endogenous thioretinaco biosynthesis, formed from thioretinamide and catalyzed by cystathionine synthase; (4) adenosyl methionine and pyridoxal phosphate to activate cystathionine synthase; (5) diallyl trisulfide or diallyl disulfide to activate cystathionine synthase, cystathionase, and hemoxygenase for the purpose of endogenous production of hydrogen sulfide; (6) amygdalin to serve as a precursor of cyanide for reaction with hydrogen sulfide to produce thiocyanate; (7) n-3 unsaturated oils to lower blood homocysteine levels; (8) pancreatic porcine enzymes and proenzymes to catabolize homocysteinylated proteins, nucleic acids and glycosaminoglycans; (9) tryptophan and mixed essential amino acids to promote biosynthesis of transthyretin; (10) menaquinone to prevent dystrophic calcification; (11) vitamin D₃ to stabilize mitochondrial thioretinaco ozonide; (12) folate to provide a substrate for biosynthesis of methyltetrahydrofolate, the coenzyme for biosynthesis of methyl cobalamin and methylation of homocysteine to methionine; (13) riboflavin as a precursor to methylenetetrahydrofolate reductase to catalyze biosynthesis of methyltetrahydrofolate and methylcobalamin for methylation of homocysteine to methionine; (14) nicotinamide riboside as a precursor to nicotinamide adenine dinucleotide, the catalyst for dehydrogenation reactions; (15) napabucasin to inhibit Stat3 signaling, thereby causing apoptosis of cancer stem cells; (16) dietary modification to eliminate processed foods and alcohol and to increase consumption of nitrilosides, proteins containing methionine, and organic sulfur compounds, such as diallyl trisulfide, diallyl disulfide, methyl allyl sulfide to promote biosynthesis of hydrogen sulfide and to convert endogenous cyanide to thiocyanate by reaction with hydrogen sulfide, facilitating homocysteine catabolism by trans-sulfiiration of homocysteine to cysteine and cysteine catabolites; (17) broad spectrum antibiotics to eliminate intracellular micro-organisms associated with formation of arteriosclerotic plaques and formation of cerebral amyloid deposition within plaques and tangles; and (18) consumption of medium chain saturated triglycerides with anti-microbial activity.

The required doses of the components of my invention are exemplified in Examples 1 through 7. However, my invention is effective over a wide range of dosage of thioretinamide, depending upon the stage and severity of the degenerative disease under treatment. The dose of thioretinamide is effective over a broad range of concentrations, from 7.5 mg/70 kg/day to 300 g/70 kg/day. Subjects with severe disease risk and symptoms are conveniently treated with a high dose of thioretinamide, 300 mg/70 kg/day, for a period of one to six months in order to halt progression of the disease. Subjects with moderate disease risk and symptoms are conveniently treated with a moderate dose of thioretinamide, 75 mg/70 kg/day, to control symptoms of disease, and subjects with a low risk of disease are conveniently treated with a low dose of thioretinamide, 7.5 mg/70 kg/day, to maintain health without disease over a period of years. Thioretinamide can also be used in combination with retinol over a wide range of dosage and over a wide range of relative amounts of the two compounds. Subjects with high disease risk and symptoms are conveniently treated with a high ratio of thioretinamide to retinol, varying from 100:1 to 1:1 on a molar basis. Subjects with low or moderate disease risk and symptoms are conveniently treated with a lower ratio of thioretinamide to retinol, varying from 1:1 to 1:100 on a molar basis. Retinol can be used effectively as a substitute for thioretinamide in subjects with low or moderate risk of disease over a wide range of dosage, varying from 7.5 mg/70 kg/day to 300 mg/7-kg/day. These wide dose ranges of thioretinamide and retinol, varying from 7.5 mg/70 kg/day to 300 mg/70 kg/day, as used in my invention, are associated with no evidence of toxicity, such as weight loss, nausea, impairment of liver function, headache, or skin lesions, in animal tests or in human trials.

The dosage of pancreatic enzymes and pro-enzymes used in my invention, as exemplified in Examples 1 through 6, is effective over a wide range of concentrations, varying from 1.4 g/70 kg/day to 14 g/70 kg/day. Subjects with high risk of disease benefit from the higher dose range, and subjects with low risk of disease or on maintenance therapy benefit from the lower dose range. The relative ratios of pancreatic enzyme dose to thioretinamide dose or retinol dose are effective over a wide range, varying from 1.4 g/70 kg/day to 14 g/70 kg/day of enzymes, from 7.5 mg/70 kg/day to 300 mg/70 kg/day for thioretinamide, and from 7.5 mg/70 kg/day to 300 g/70 kg/day for retinol. The doses of the vitamins, amino acids, nitrilosides, n-3 unsaturated oils, broad spectrum antibiotics, medium chain saturated triglycerides, diallyl trisulfide, diallyl disulfide, methyl allyl sulfide, and other allyl sulfides, napabucasin, and other furanonaphthoquinones, are similarly effective over a broad range of dosage, as exemplified in Examples 1 through 6. The following ranges of doses of these components of my invention are useful from controlling disease risk by my invention: ascorbate 0.1 to 10 g/70 kg/day; cobalamin 0.01 to 1 mg/70 kg/day, adenosyl methionine, 0.1 to 1.0 g/70 kg/day; pyridoxal, 2.0 to 200 g/70 kg/day; amygdalin, 0.1 to 10 g/70 kg/day; n-3 unsaturated oils, 1.0 to 20 g/70 kg/day; tryptophan and mixed essential amino acids, 0.1 to 1.0 g/70 kg/day; folate 0.2 to 2.0 mg/70 kg/day; riboflavin, 1.0 to 50 mg/70 kg/day; nicotinamide riboside, 1.0 to 50 mg/70 kg/day; diallyl trisulfide, 10 to 300 mg/70 kg/day; napabucasin, 10 to 300 mg/70 kg/day; menaquinone, 0.01 to 1.0 mg/70 kg/day; vitamin D₃ 100 to 2000 IU/70 kg/day; folate, 0.2 to 2.0 mg/70 kg/day; broad spectrum antibiotics such as doxycycline or minocycline, 0.1 to 5.0 g/70 kg/day; medium chain saturated triglycerides, 1.0 to 10 g/70 kg/day.

The novel method for utilization of thioretinamide by the metabolic protocol embodied in my invention is useful for prevention of induction malignant neoplasms and for treatment of primary and metastatic neoplasms in human subjects exposed to carcinogenic chemicals, radiation, or oncogenic microbes, causing regression of malignant cell proliferation. The method of my invention is also useful in prevention and regression of arteriosclerotic plaques of aorta and peripheral arteries in human subjects exposed to an atherogenic diet and multiple infections of the plaques by pathogenic micro-organisms. The method of my invention is also useful for prevention and regression of cerebral plaques and tangles within neurons in subjects with dementia that are exposed to multiple infections by pathogenic micro-organisms. The method of my invention is also useful in prevention of the replication of pathogenic viruses to prevent or cause regression of the pathogenic effects of these viruses and to prevent post-infection sequelae of these viruses. The method of my invention is also useful in preventing the degenerative aging changes of their tissues, decreased oxidative metabolism, and decreased life expectancy associated with aging by prevention of further degenerative changes of tissues associated with aging, by enhancement of oxidative metabolism, and by prolongation of life span. In this respect the method of my invention is non-toxic and does not suffer the drawback of many known anti-neoplastic, anti-atherogenic, anti-viral, and anti-aging agents, which have cumulative toxic effects after prolonged administration.

Currently available therapies for acute coronary syndrome are not totally effective in preventing recurrent adverse vascular disease events. Current therapies with anti-platelet agents, beta-blockers, anticoagulants, thromboplastin activators, calcium channel inhibitors, and angiotensin converting enzyme inhibitors are only partially effective in therapy. Treatment of the hyperhomocysteinemia associated with arteriosclerosis with pyridoxal, folate, and cobalamin does not prevent recurrence of adverse vascular events in subjects with advanced cardiovascular, cerebrovascular, or peripheral vascular disease. Treatment of subjects with early cognitive decline with pyridoxal, folate and cobalamin prevents shrinkage of cerebral tissue susceptible to dementia. However, therapy to remove amyloid deposits from cerebral plaques and intracellular tangles is ineffective in preventing further cognitive decline and dementia. Treatment of human subjects with the method of my invention will correct the underlying metabolic abnormality leading to acute coronary syndrome and other forms of vascular disease, including vascular dementia, by restoring depleted concentrations of thioretinamide and thioretinaco ozonide within mitochondrial membranes of vascular cells and neurons, restoring endothelial function, preventing a prothrombotic state, and restoring nitric oxide function. The anti-microbial effects of diallyl trisulfide, napabucasin, proteolytic enzymes, antibiotics, and medium chain saturated mono-glycerides of my invention will retard and prevent growth of micro-organisms associated with the pathogenesis of arteriosclerosis and dementia. The hyperhomocysteinemia that is characteristic of acute coronary syndrome, metabolic syndrome, chronic arteriosclerosis, and dementia will be prevented by restoration of mitochondrial thioretinaco ozonide, preventing vascular injury, endothelial dysfunction, progression of arteriosclerotic plaques, progression of cerebral plaques and tangles, and recurrent adverse vascular events, such as coronary thrombosis, myocardial infarction, cerebrovascular thrombosis, cerebral infarction, and ischemic gangrene of the extremities.

The novel method of utilization of thioretinamide of my invention is deemed useful in preventing the occurrence of spontaneous human neoplasms, including, but not limited to, cancer of lung, skin, colon, breast, prostate, pancreas, brain, lymph nodes, liver, kidney or other organs that arise because of exposure to carcinogenic chemicals, electromagnetic radiation, radiation from radioactive elements, viruses, micro-organisms, inflammatory cytokines, dietary factors, or genetic factors. My invention is further deemed useful for the treatment of human neoplasms, causing regression of or preventing metastasis of malignant neoplasms. It is also deemed that this invention is useful in treatment of human atherosclerosis, involving aorta, coronary, renal, peripheral, cerebral or other major arteries, causing regression of and prevention progression of arteriosclerotic plaques, thereby preventing or ameliorating coronary heart disease, stroke, renovascular disease, and peripheral vascular disease. My invention is also deemed useful in treatment of human pathogenic virus infections, including, but not limited to hepatitis virus, immune-deficiency virus, hemorrhagic fever viruses, encephalitis viruses, influenza virus, rhinoviruses, pox viruses, herpetic viruses, and enteric viruses, by preventing viral replication and spread of the virus infection within the cells of the various tissues of the body. My invention is also deemed useful in treatment of human degenerative diseases associated with aging, including, but not limited to, osteoarthritis, osteoporosis, cataract, macular degeneration, dementia, diabetes mellitus, metabolic syndrome, rheumatoid arthritis, thyroiditis, lupus erythematosus, pernicious anemia, and other autoimmune disorders, causing remission or preventing of progression of these diseases within the tissues of the body. It is expected that my invention will be useful in prolonging human life span by preventing degenerative diseases of aging, including atherosclerosis, cancer, autoimmune diseases, and age-associated loss of function of brain, heart, lungs, liver, kidneys, eyes, ears, and other major organs.

The range of useful concentrations of thioretinamide is broad, extending from 0.1-60 mg/kg of body weight. The subject invention can be administered to human subjects in the aforesaid dosage range. Suspensions, emulsions and dispersions of thioretinamide can be administered by the enteric route, employing capsules and time-release formulations, mixed with suitable inert carriers. The subject invention can also be administered parenterally in compatible solvents and vehicles, given intravenously, intramuscularly, introperitoneally, subcutaneously, intracisternally, intrathecally, and within neoplasms in various internal organs by direct injection, with ultrasound, nuclear magnetic resonance, or X-ray computerized tomography guidance. The administration of thioretinamide is combined with the other supportive measures, previously enumerated, to maximize the therapeutic efficacy of the treatments.

It will be understood by those skilled in the art that the actual preferred amount of thioretinamide used will vary according to the specific isomer being used, the particular compositions formulated, the mode of application and particular site and subject being treated. Optimal application rates for a given set of conditions can be ascertained by those skilled in the art, using conventional dosage determination tests in accordance with the detailed description of this invention.

The advantages of my invention, as well as aspects of the preferred embodiments, are illustrated more fully in the following Examples:

### EXAMPLE 1

A 75 year old man was evaluated for treatment of metastatic prostate cancer. The prostate specific antigen (PSA) of blood was determined at 8.0 ng/mL, and needle biopsy demonstrated well differentiated adenocarcinoma, Gleason grade 3+3=6/10, Following radical prostatectomy, the PSA value was 0.1 ng/mL and gradually increased to 8.0 ng/mL over a period of 2 years. The plasma homocysteine was initially 10.8 μmol/L, gradually rising to 14.0 μmol/L over a period of 2 years. Computerized tomography scan demonstrated enlarged retroperitoneal lymph nodes, and biopsy of the prostatic surgical site demonstrated recurrent adenocarcinoma. The bone scan revealed no evidence of metastasis. Following luprolide therapy, the PSA value declined to 0.1 ng/mL over a period of three months. To prevent metastasis and hormone resistance of the adenocarcinoma, luprolide therapy was discontinued, and a metabolic protocol was employed. The oral medications and supplements consisted of synthetic thioretinamide, 75 mg per day; pancreatin, 1.4 g four times per day; diallyl trisulfide, 300 mg per day; napabucasin, 300 mg per day; amygdalin, 500 mg per day; adenosyl methionine, 200 mg per day; pyridoxal phosphate, 25 mg per day; cyanocobalamin, 500 μg sublingual per day; ascorbate with mixed bioflavonoids, 1000 mg per day; folic acid, 800 μg per day; riboflavin, 25 mg per day; nicotinamide riboside, 125 mg per day; menoquinone, 90 μg per day; vitamin D₃, 2000 units per day; fish oil, 3 g per day; cod liver oil, 15 mL per day; tryptophan 500 mg per day; mixed amino acids 150 mg each per day. The diet was adjusted to eliminate all processed foods containing sugar, white flour, powdered milk, powdered eggs, protein hydrolyzates, natural flavoring extracts, and alcohol. Dietary consumption of berries, nuts, apricot seeds, beans, molasses, fruits, raw vegetables, steamed vegetables, fish and fresh meats was increased, After following the metabolic protocol for one year the PSA value was 0.1 ng/mL, the plasma homocysteine was 10.4 μmol/L, and the repeat CT scan and bone scan showed no evidence of lymphadenopathy or bone metastasis. At a subsequent visit 5 years later, the PSA value was 0.1 ng/mL, the plasma homocysteine was 9.8 μmol/L, and no weight loss or pain were reported, while continuing the metabolic protocol.

### EXAMPLE 2

A 75 year old woman was evaluated for treatment of macular degeneration and mild recent memory loss. Three years previously decreased vision was noticed in the left eye, and ophthalmological evaluation revealed early supranuclear cataracts bilaterally with edema of the macular area on the left, associated with drusen and retinal pigment epithelium changes. The plasma homocysteine level was 15.4 mol/L, and the plasma hs-C-reactive protein (CRP) was 3.2 μmol/mL. The Mini Mental State Examination (MMSE) value was 26.6/30, revealing mild cognitive impairment. The woman never smoked, but there was a family history of macular degeneration. After 3 years, she returned with decreased vision in the right eye, and examination revealed macular edema associated with drusen and retinal pigment epithelium changes. Dental examination revealed caries, plaque, and extensive periodontitis. To prevent progression of macular changes and decline in mental function, a metabolic protocol was employed. The oral medications and supplements consisted of synthetic thioretinamide, 75 mg per day; retinol, 20,000 IU per day as synthetic retinol or from cod liver oil, 15 mL per day; pancreatin, 1.4 g four times per day; diallyl trisulfide, 300 mg per day; napabucasin, 300 mg per day; amygdalin, 100 mg per day; adenosyl methionine, 200 mg per day; pyridoxal phosphate, 25 mg per day; cyanocobalamin, 500 μg sublingual per day; ascorbate with mixed bioflavonoids, 1000 mg per day; folic acid, 800 μg per day; riboflavin, 25 mg per day; nicotinamide riboside, 125 mg per day; menoquinone, 90 μg per day; vitamin D₃, 2000 IU per day; fish oil, 3 g per day; tryptophan, 500 mg per day; mixed amino acids, 150 mg each per day; monolaurin, as coconut oil, 60 g per day; doxycycline, 100 mg per day. The diet was adjusted to eliminate all processed foods containing sugar, white flour, powdered milk, powdered eggs, protein hydrolyzates, natural flavoring extracts, and alcohol. Dietary consumption of berries, nuts, beans, molasses, fruits, raw vegetables, steamed vegetables, fish and fresh meats was increased. Professional dental hygiene was provided every 3 months. After following the metabolic protocol for one year, the plasma homocysteine was 10.7 μmol/L, and the CRP was 0.5 μmol/mL. The repeat MMSE was 28.5/30. Visual acuity did not change, and examination revealed decreased macular edema bilaterally. Improved memory was reported by her husband. At a subsequent visit 2 years later, the plasma homocysteine was 8.9 μmol/L, the CRP was less than 0.5 μmol/mL, the MMSE was 28.0/30, visual acuity and macular appearance were unchanged, while continuing the protocol.

### EXAMPLE 3

A 60 year old man was admitted to hospital with intermittent chest pain and shortness of breath. The man was diaphoretic and restless with acute distress. The troponin was 1.5 ng/mL, the white blood cell count was 15,000/mm³, the plasma homocysteine was 15.8 μmol/L, and the CRP was 7.5 μmol/mL. The electrocardiogram demonstrated ST elevation in the precordial leads. A chest X-ray showed early pulmonary edema and congestion of pulmonary arteries. Dental examination revealed caries, plaque and extensive peri-odontitis. After treatment with pain medication, bed rest, and digoxin, the electrocardiogram reverted to normal, his symptoms improved, and a metabolic protocol was employed. The oral medications and supplements consisted of synthetic thioretinamide, 75 mg per day; pancreatin, 1.4 g four times per day; diallyl trisulfide, 300 mg per day; napabucasin, 300 mg per day; amygdalin, 500 mg per day; adenosyl methionine, 200 mg per day; pyridoxal phosphate, 25 mg per day; cyanocobalamin, 500 μg sublingual per day; ascorbate with mixed bioflavonoids, 1000 mg per day; folic acid, 800 μg per day; riboflavin, 25 mg per day; nicotinamide riboside, 125 mg per day; menoquinone, 90 μg per day; vitamin D₃, 2000 units per day; fish oil, 3 g per day; cod liver oil, 15 mL per day; tryptophan 500 mg per day; mixed amino acids 150 mg each per day; monolaurin, as coconut oil, 60 g per day; doxycycline, 100 mg per day. The diet was adjusted to eliminate all processed foods containing sugar, white flour, powdered milk, powdered eggs, protein hydrolyzates, natural flavoring extracts, and alcohol. Dietary consumption of berries, nuts, beans, molasses, fruits, raw vegetables, steamed vegetables, fish and fresh meats was increased. Professional dental hygiene was provided every 3 months. After following the metabolic protocol for one year, the plasma homocysteine was 9.8 μmol/L, the CRP was 0.5 μmol/mL, and the troponin was undetectable. There was no recurrence of chest pain, and the electrocardiogram was normal. At a subsequent visit 5 years later the plasma homocysteine was 10.5 μmol/L, the CRP was less than 0.5 μmol/L, the troponin was undetectable, the electrocardiogram was normal, and there was no recurrence of chest pain, while continuing the metabolic protocol.

### EXAMPLE 4

A 65 year old male was evaluated for treatment of obesity, hypertension, and elevated blood glucose. During the previous 5 years, gradual weight gain involved abdominal viscera with a protuberant abdomen, the girth increasing to 44 inches. The blood pressure was 180 systolic and 110 diastolic. The plasma homocysteine was 16.5 μmol/L, the fasting blood glucose was 125 mg/dL, the urinalysis revealed microalbuminuria of 2.5 mg/dL, and the plasma creatinine was 2.0 mg/dL. To prevent progression of the metabolic syndrome and early renal failure, dietary improvement, moderate exercise, and a metabolic protocol were employed. The oral medications and supplements consisted of synthetic thioretinamide, 75 mg per day; retinal, 20,000 IU, as synthetic retinol or from cod liver oil, 15 mL per day; pancreatin, 1.4 g four times daily; diallyl trisulfide, 300 mg per day; napabucasin, 300 mg per day; amygdalin, 100 mg per day; adenosyl methionine, 200 mg per day; pyridoxal phosphate, 25 mg per day; cyanocobalamin, 500 μl sublingual per day; ascorbate with mixed bioflavonoids, 1000 mg per day; folic acid 800 μg per day; riboflavin, 25 mg per day; nicotinamide riboside, 125 mg per day; menoquinone, 90 μg per day; vitamin D₃, 2000 IU per day; fish oil, 3 g per day; tryptophan, 500 mg per day; mixed amino acids, 150 mg each per day; monolaurin, as coconut oil, 60 g per day. The diet was adjusted to eliminate all processed foods containing sugar, white flour, powdered milk, powdered eggs, protein hydrolyzates, natural flavoring extracts, and alcohol. Dietary consumption of berries, nuts, beans, molasses, fruits, raw vegetables, steamed vegetables, fish and fresh meats was increased. Moderate exercise, consisting of a 1.5 mile walk three days per week, and doubles tennis once per week were employed. After following the dietary adjustment, exercise program and metabolic protocol for one year, the plasma homocysteine was 11.0 μmol/L, the fasting blood glucose was 98 mg/dL, the urinalysis revealed no protein, and the plasma creatinine was 1.5 mg/dL. Weight loss of approximately 15 pounds was reported, and the abdominal girth measured 41 inches. The blood pressure was 140 systolic and 85 diastolic. At a subsequent visit 2 years later, additional weight loss of 10 pounds and a girth of 40 inches were reported, while continuing the metabolic protocol. The plasma homocysteine was 10.2 mol/L, blood glucose was 95 mg/dL, the urinalysis revealed no protein, and the plasma creatinine was 1.7 mg/dL. The blood pressure was 140 systolic and 85 diastolic.

### EXAMPLE 5

A 70 year old man with mild abdominal pain was evaluated for treatment of an abdominal aortic aneurysm that was detected by computerized tomography. On examination, a 1 cm ulcer was found on the right great toe. The patient reported the onset of pain in the lower extremities after walking approximately 50 yards. The plasma homocysteine was 18.5 μmol/L, the CRP was 10.7 μmol/mL, and the fasting blood glucose was 98 mg/dL. Dental examination revealed caries, plaque, and extensive peri-odontitis. Surgical treatment consisted of excision of the abdominal aortic aneurysm with grafting of the distal aorta, followed by endarterectomy of the right common femoral artery with grafting. The pathology report confirmed the presence of an arteriosclerotic aortic aneurysm, with laminated mural thrombus, and inflammatory changes of the adventitia. Also, fibro-calcific arteriosclerotic plaques were found in the common femoral artery with severe narrowing of the lumen. After recovery from surgery, the ulceration of the great toe gradually healed spontaneously. To prevent progression of generalized arteriosclerosis, a metabolic protocol, dietary adjustment, and antibiotic therapy were employed. The oral medications and supplements consisted of synthetic thioretinamide, 75 mg per day; pancreatin, 1.4 g four times per day; diallyl trisulfide, 300 mg per day; napabucasin, 300 mg per day; amygdalin, 100 mg per day; adenosyl methionine, 200 mg per day; pyridoxal phosphate, 25 mg per day; cyanocobalamin, 500 pg sublingual per day; ascorbate with mixed bioflavonoids, 1000 mg per day; folic acid 800 μg per day; riboflavin, 25 mg per day; nicotinamide riboside, 125 mg per day; menoquinone, 90 μg per day; vitamin D₃, 2000 IU per day; fish oil, 3 g per day; cod liver oil, 15 mL per day; tryptophan, 500 mg per day; mixed amino acids, 150 mg each per day; monolaurin, as coconut oil, 60 g per day; doxycycline, 100 mg per day. The diet was adjusted to eliminate all processed foods containing sugar, white flour, powdered milk, powdered eggs, protein hydrolyzates, natural flavoring extracts, and alcohol. Dietary consumption of berries, nuts, beans, molasses, fruits, raw vegetables, steamed vegetables, fish and fresh meats was increased. Professional dental hygiene was provided every 3 months. After following the dietary adjustment and metabolic protocol for one year, the plasma homocysteine was 12.5 μmol/L, the CRP was 1.5 μmol/mL, and the fasting blood glucose was 95 mg/dL. There was no recurrence of the ulcer of the great toe, and there were no symptoms of intermittent claudication. At a subsequent examination 2 years later, the plasma homocysteine was 10.8 μmol/L, the CRP was 1.2 μmol/mL, and the fasting blood glucose was 95 mg/dL. There were no further symptoms of abdominal pain, skin ulcers or intermittent claudication, while following the metabolic protocol.

### EXAMPLE 6

A 65 year old woman was evaluated for the sudden onset of right sided weakness, associated with inability to speak and difficulty seeing objects in the right visual field. The plasma homocysteine was 18.0 μmol/L, the CRP was 12.5 μmol/mL, and the fasting blood glucose was 102 mg/dL. Following thrombolytic therapy for stroke, her symptoms gradually improved while convalescing at home. After recovering for 3 months, she tripped on a rug and fell, fracturing the right femoral neck. After surgical fixation of the fracture, she was evaluated for osteoporosis that was demonstrated on the X-rays of her fracture site and spine. To prevent further episodes of cerebrovascular disease and fracture, dietary adjustment and a metabolic protocol were employed. The oral medications and supplements consisted of synthetic thioretinamide, 75 mg per day; pancreatin, 1.4 g four times per day; diallyl trisulfide, 300 mg per day; napabucasin, 300 mg per day; amygdalin, 100 mg per day; adenosyl methionine, 200 mg per day; pyridoxal phosphate, 25 mg per day; cyanocobalamin, 500 μg sublingual per day; ascorbate with mixed bioflavonoids, 1000 mg per day; riboflavin, 25 mg per day; nicotinamide riboside, 125 mg per day; menoquinone, 90 μg per day; vitamin D₃, 2000 IU per day; fish oil, 3 g per day; cod liver oil, 15 mL per day; tryptophan, 500 mg per day; mixed amino acids, 150 mg each per day; monolaurin, as coconut oil, 60 g per day; doxycycline, 100 mg per day. The diet was adjusted to eliminate all processed foods containing sugar, white flour, powdered milk, powdered eggs, protein hydrolyzates, natural flavoring extracts, and alcohol. Dietary consumption of berries, nuts, beans, molasses, fruits, raw vegetables, steamed vegetables, fish and fresh meats was increased. After following the dietary adjustment and metabolic protocol for one year, the plasma homocysteine was 12.5 μmol/L, the CRP was 4.5 μmol/mL, and the fasting blood glucose was 86 mg/dL. The hemi-paresis and visual field defects were no longer demonstrated. There were no further episodes of mental changes, visual disturbances, or weakness. The hip prosthesis was satisfactory, permitting full ambulation. At a subsequent evaluation after 2 years, the plasma homocysteine was 10.5 μmol/L, the CRP was 0.5 μmol/mL, and the fasting blood glucose was 85 mg/dL. There were no further symptoms of weakness, visual disturbances, or mental changes, and ambulation was satisfactory, while following the metabolic protocol.

### EXAMPLE 7

Human adenocarcinoma cells were cultured in RPMI medium with added fetal bovine serum and antibiotics, as reported by McCully K S et al in Research Communications in Chemical Pathology and Pharmacology, 1992; 77: 125-128 and reviewed by Ariga T et al in Biofactors 2006; 26: 93-103. Equal numbers (10⁵) of adenocarcinoma cells were trypsinized and passaged on day 0, and the cells were refed with media containing test compounds on days 1 and 3. Cell numbers were determined by trypsinization on day 4. Thioretinaco (TR₂Co) and thioretinamide (TR) were synthesized, as previously described (McCully U.S. Pat. No. 4,618,685; McCully U.S. Pat. No. 4,925,931) and dissolved in absolute ethanol. Authentic diallyl trisulfide (FATS) and napabucasin (NBS) were solubilized in propylene glycol. Propylene glycol (PG) was added to the ethanol solutions of the test compounds, the ethanol was evaporated under reduced pressure at 37° C., and the resulting solutions in propylene glycol were added to the culture media.

TABLE 2
Effect of thioretinaco, thioretinamide, diallyl trisulfide, and
napabucasin on growth of cultured human adenocarcinoma cells.

| | Concentration | cell growth | cell growth |
|---|---|---|---|
| Addition to media | (mg/dL) | (10⁵) | (% of control) |
| None | — | 7.4 ± 2.7 | 100 |
| TR₂Co | 30 | 2.5 ± 0.82 | 33 |
| TR | 30 | 1.4 ± 0.50 | 19 |
| DATS | 1.0 | 2.7 ± 0.57 | 36 |
| NBS | 1.0 | 2.3 ± 0.52 | 28 |
| TR₂Co + DATS | 30 + 1.0 | 0.5 ± 0.06 | 6.7 |
| TR₂Co + NBS | 30 + 1.0 | 0.4 ± 0.05 | 4.8 |
| TR₂Co + DATS + NBS | 30 + 1.0 + 1.0 | 0.2 ± 0.05 | 2.7 |
| TR + DATS | 30 + 1.0 | 0.7 ± 0.08 | 9.5 |
| TR + NBS | 30 + 1.0 | 0.5 ± 0.06 | 4.5 |
| TR + DATS + NBS | 30 + 1.0 + 1.0 | 0.3 ± 0.06 | 3.7 |
| PG | 500 | 9.1 ± 0.80 | 123 |

As demonstrated in Table 2, thioretinaco, thioretinamide, diallyl trisulfide, and napabucasin each significantly inhibit growth of cultured human adenocarcinoma cells. Addition of diallyl trisulfide, diallyl trisulfide and napabucasin to thioretinaco and to thioretinamide causes a synergistic inhibition of growth of cultured human adenocarcinoma cells.

My invention is useful in therapy of human diseases characterized by impairment of both the immune system in increasing the apoptosis of malignant cells and in utilizing thioretinamide and homocysteine catabolism, as reflected in elevation of blood homocysteine concentrations. From the foregoing description, one skilled in the art can easily ascertain the essential characteristics of my invention, and, without departing from the spirit and scope thereof, can make various changes and modifications to adapt my invention to various usages and conditions.

1. A composition of matter to treat cancer by stimulating the immune system and increasing the apoptosis of malignant cells comprising a therapeutically effective amount of thioretinamide, a therapeutically effective amount of ally! sulfide and optionally a therapeutically effective amount of pancreatin.

2. The composition of claim 1 wherein the therapeutically effective amount of allyl sulfide is a therapeutically effective amount of diallyl trisulfide.

3. The composition of claim 1 wherein the composition includes a therapeutically effective amount of pancreatin.

4. The composition of claim 1 further comprising a therapeutically effective amount of cobalamin.

5. The composition of claim 1 further comprising a therapeutically effective amount of pyridoxal.

6. The composition of claim 1 further comprising a therapeutically effective amount of folate.

7. The composition of claim 1 further comprising a therapeutically effective amount of adenosyl methionine.

8. A composition of matter to treat cancer by stimulating the immune system and increasing the apoptosis of malignant cells comprising in combination a therapeutically effective amount of thioretinamide, a therapeutically effective amount of a furanonaphthoquinone and optionally a therapeutically effective amount of pancreatin to stimulate the immune system and cross the blood brain barrier.

9. The composition of claim 8 wherein the therapeutically effective amount of the furanonaphthoquinone is a therapeutically effective amount of napabucasin.

10. The composition of claim 8 wherein the composition includes the therapeutically effective amount of pancreatin.

11. The composition of claim 8 further comprising a therapeutically effective amount of cobalamin.

12. The composition of claim 8 further comprising a therapeutically effective amount of pyridoxal.

13. The composition of claim 8 further comprising a therapeutically effective amount of folate.

14. The composition of claim 8 further comprising a therapeutically effective amount of adenosyl methionine.

15. A method of treating cancer including glioblastoma with a blood brain barrier penetrating composition to induce apoptosis of malignant cells comprising a therapeutically effective amount of thioretinamide, a therapeutically effective amount of allyl sulfide, a therapeutically effective amount of furanonaphthoquinone and optionally a therapeutically effective amount of pancreatin.

16. The method of claim 15 wherein the therapeutically effective amount of allyl sulfide is diallyl trisulfide.

17. The method claim 15 wherein the therapeutically effective amount of furanonaphthoquinone is napabucasin.

18. The method of claim 15 further comprising a therapeutically effective amount of cobalamin.

19. The method of claim 15 wherein the composition includes the therapeutically effective amount of pancreatin.

20. The method of claim 15 further comprising a therapeutically effective amount of retinol.

21. The composition of claim 8 further comprising a therapeutically effective amount of an allyl sulfide.

22. The composition of claim 21 wherein the therapeutically effective amount of the allyl sulfide is diallyl trisulfide.

23. A composition of matter to treat cancer and increase the apoptosis of malignant cells comprising a therapeutically effective amount of thioretinamide, a therapeutically effective amount of a furanonaphthoquinone and optionally a therapeutically effective amount of pancreatin to penetrate the blood brain barrier.

24. The composition of claim 23 wherein the therapeutically effective amount of the furanonaphthoquinone is napabucasin.

25. The composition of claim 23 further comprising a therapeutically effective amount of an allyl sulfide.

26. The composition of claim 25 wherein the therapeutically effective amount of the allyl sulfide is diallyl trisulfide.

27. The composition of claim 23 wherein the composition includes the therapeutically effective amount of pancreatin.

28. The composition of claim 23 further comprising a therapeutically effective amount of cobalamin.

29. The composition of claim 23 further comprising a therapeutically effective amount of pyridoxal.

30. A composition of matter to treat cancer comprising a therapeutically effective amount of about 7.5 to 300 mg/70 kg/day of thioretinamide, a therapeutically effective amount of about 10 to 300 mg/70 kg/day of a furanonaphthoquinone and optionally a therapeutically effective amount of pancreatin.

31. The composition of claim 30 wherein the therapeutically effective amount of the furanonaphthoquinone is about 10 to 300 mg/70 kg/day of napabucasin.

32. The composition of claim 30 further comprising a therapeutically effective amount of about 1.0 to 50 mg/70 kg/day of diallyl trisulfide.
